(19) United States
(12) Patent Application Publication
(10) Pub. No.: US 2008/0124776 A1
(43) Pub. Date: May 29, 2008

(54) Carbodithioate Ligands For Nanotechnology and Methods of Use

(76) Inventor: Alexander Wei, West Lafayette, IN (US)

Correspondence Address: BOSE MCKINNEY & EVANS LLP;JAMES COLES, 135 N PENNSYLVANIA ST, SUITE 2700, INDIANAPOLIS, IN, 46204, US

(21) Appl. No.: 11/853,001

(22) Filed: Sep. 10, 2007

Related U.S. Application Data
(63) Continuation-in-part of application No. 11/407,751, filed on Apr. 20, 2006.
(60) Provisional application No. 60/673,190, filed on Apr. 20, 2005.

(51) Int. Cl. C12N 13/00; A61N 5/06

(52) U.S. Cl. 435/173.1; 604/20

(57) ABSTRACT

The present invention is directed to methods and products related to carbodithioate ligands bonded to surfaces. The invention is further directed to methods of inducing cell injury or death. Such methods may be useful in the treatment of diseases such as cancer.

This application is a continuation-in-part of U.S. patent application Ser. No. 11/407,751 filed Apr. 20, 2006, which claims priority to U.S. Provisional Patent Application 60/673,190 filed on Apr. 20, 2005, both of which are incorporated by reference in their entirety herein.

### GOVERNMENT RIGHTS

This invention was made with government support under grant reference numbers NSF CHE-0243496 and ECS-0210445 awarded by the National Science Foundation and EB-001777-01 and GM-06982-01 awarded by the National Institutes of Health. The Government may have certain rights in the invention.

### BACKGROUND OF THE INVENTION

The present invention relates to methods for preparing carbodithioate ligands bonded to surfaces, methods for biosensing with carbodithioate ligands bonded to surfaces, and carbodithioate ligands.

The functionalization of surfaces with organic ligands has become an important aspect of surface science and nanomaterials chemistry. For example, molecular monolayers are often formed spontaneously by methods, such as the self-assembly of alkanethiols on gold for preparing surfaces, with tunable physical or chemical properties or with molecular recognition elements. Such self-assembled monolayers (SAMs) have potential utility as biosensors for molecules that bind to them such as peptides, small proteins, DNA, carbohydrates, oligonucleotides, and bioactive natural products.

However, SAMs are often limited by their chemical stability. By way of example, thiols, which are often used as the organic ligands for SAMs, can be readily oxidized to disulfides or sulfonates and can desorb or be replaced from the surface by other molecules for being incompatible with other functional groups associated with the ligand. Because they are not sufficiently stable in biological fluids, such thiol-based SAMs have been shown to lack the long-term stability needed for most biomedical applications such as biosensing, which include the use of compounds to detect molecules of biological interest. As such, it would be desirable to have a surface functionalization method that is sufficiently robust for utility in biomedical applications.

### SUMMARY OF THE INVENTION

The current invention advantageously provides methods for preparing robust carbodithioate ligands bonded to surfaces. Ligands bearing the —CS₂ group, for example, have superior chemisorption properties than thiols on a number of surfaces and are more stable in fluids under biologically relevant conditions.

With respect to SAMs that are nanoparticles, metal nanoparticles (e.g., gold nanorods), semiconductor nanoparticles (e.g., CdSe “quantum dot” nanocrystals), and superparamagnetic nanoparticles have excellent potential as site-directed contrast agents in biomedical imaging. The targeted delivery of these nanoparticles to particular regions of the body is dependent on a robust method of surface functionalization, to maintain appropriate levels of biodistribution and to prevent nonspecific cell uptake or protein adsorption. Thiols are inadequate for maintaining stable passivation on metal surfaces, but dithiocarbamates and other carbodithioates are much more robust and will resist surface desorption or displacement under biologically relevant conditions. The robustness of carbodithioate-anchored ligands is also useful for the directed delivery of nanoparticle agents to diseased tissue.

In one aspect of the invention, methods for preparing a surface bonded to a carbodithioate ligand comprising treating the surface with a mixture comprising a nucleophile and a sulfur-containing compound in a suitable solvent are provided.

The invention also relates in one aspect to processes for functionalizing a surface comprising treating a surface bonded to a sulfur atom with a sulfur-bearing compound selected from an isothiocyanate and R²⁰NCS, where R²⁰ is independently selected from alkyl, aryl or heteroaryl.

The invention further provides methods for preparing carbodithioate ligands comprising suspending a surface in an aqueous medium and treating the surface with a sulfur-containing compound and a nucleophile.

In yet a further aspect of the invention, carbodithioate ligands alone and bonded with surfaces are provided.

Another aspect of the invention is directed to methods for detecting a molecular or biomolecular analyte using a surface-bound carbodithioate ligand as a recognition element.

Yet another aspect of the invention is directed to methods for functionalizing a surface comprising passivating a surface with a mixture of a carbodithioate ligand not used for molecular recognition and a carbodithioate ligand capable of molecular recognition.

Another aspect of the invention is directed to methods for preparing a core-shell nanomaterial comprising encapsulating a nanomaterial with a carbodithioate ligand; extracting the encapsulated nanomaterial into an organic solvent; and treating with an organometallic compound to form a core-shell nanomaterial.

An additional aspect of the invention is directed to methods for preparing a nanoparticle imaging agent comprising passivating the surface of a nanoparticle and functionalizing the nanoparticle surface with a combination of biologically active nucleophiles and biologically inactive nucleophiles.

A further aspect of the invention is directed to methods for inducing cell death in tumor cells using a nanomaterial functionalize with a biologically functional ligand.

Yet a further aspect of the invention is directed to methods for the treatment of mammals having tumors using a nanomaterial with a biologically functional ligand.

The above-mentioned aspects of the present invention and the manner of obtaining them will become more apparent and the invention itself will be better understood by reference to the following description of the embodiments of the invention taken in conjunction with the accompanying drawings.

### BRIEF DESCRIPTION OF THE DRAWINGS

FIG. 1 depicts dithiocarbamate ligands formed on Au surfaces with several exemplary ligand examples in accordance with the present invention; and

FIG. 2 depicts selected SERS spectra of dialkyl DTCs formed on roughened Au surfaces obtained using a dispersive Raman microscope with a 20× objective lens (N.A.=0.4) at an excitation wavelength of 785 nm and an exposure time of 30 seconds in accordance with the present invention.

### DETAILED DESCRIPTION OF THE INVENTION

The embodiments of the present invention described below are not intended to be exhaustive or to limit the invention to the precise forms disclosed in the following detailed description. Rather, the embodiments are chosen and described so that others skilled in the art may appreciate and understand the principles and practices of the present invention.

In one embodiment of the invention, the carbodithioate ligands of the invention are prepared in a mixture of a sulfur-containing compound and a nucleophile in a suitable solvent. It is understood that molecules bearing the carbodithioate ligand may deliver the ligand to the surface. A surface is treated with this mixture by, for example, immersing the surface into the mixture. The carbodithioate ligand may be bonded to the surface by condensing the mixture in the suitable solvent onto the surface. In one preferred embodiment, the approximate molar ratio of the sulfur-containing compound to the nucleophile is 1.

In another embodiment of the invention, the sulfur-containing compound is CS₂ and is treated with a nitrogen-containing nucleophile to form a carbodithioate ligand bonded to a surface wherein the carbodithioate ligand is selected from R¹R²N—CS₂; xanthates; R⁹O—CS₂; R¹⁰SCS₂; or R¹¹R¹²P(═O)—CS₂ wherein R₁ and R₂ are independently selected from —H, alkyl, acyl, aryl, heteroaryl, —OR³, —NR⁴R⁵, SiR⁶R⁷R⁸ or SR⁹;

R³, R⁴, R⁵, R⁶, R⁷, R⁸, are independently selected from —H, alkyl, acyl, aryl or heteroaryl;

R⁹ is aryl or heteroaryl;

R¹⁰ is alkyl, acyl, aryl, or heteroaryl;

R¹¹ and R¹² are independently selected from alkyl, aryl, —OR¹³, —NR¹⁴R¹⁵, SiR¹⁶R¹⁷R¹⁸, or SR¹⁹;

R¹³, R¹⁴, R¹⁵, R¹⁶, R¹⁷, R¹⁸ and R¹⁹ are independently selected from —H, alkyl, acyl, aryl or heteroaryl.

In an additional embodiment of the invention, a preformed carbodithioate ligand is R²⁰R²¹NCS₂—S₂CNR^{20′}R^{21′} wherein R^{20′} and R^{21′} are independently selected from H, alkyl, acyl, aryl or heteroaryl.

The surface of the invention may be inorganic or metal. A preferred surface is gold. Examples of metals of the invention include Group 13 metals, group 14 metals, and group 15 metals. Other surfaces of the invention include iron oxide, cadmium selenide, cadmium sulfide, and indium tin oxide. Surfaces of the invention may also be magnetic in nature such as, but not limited to, cobalt ferrite.

When the surface of the invention is metal, it may be selected from thiophilic metal surfaces including gold, silver, copper, palladium, platinum, and selected steel alloys. A preferred surface is gold.

Surfaces of the invention include nanoparticles. By “nanoparticle” what is meant is a particle having one or more dimensions on the order of 200 nm or less. In one embodiment of the invention, the nanoparticle has a length in one or more dimensions on the order of between about 2 nm to about 100 nm. In another embodiment of the invention, the nanoparticle has a length in one or more dimensions on the order of about 40 nm. Preferred nanoparticles are thiophilic metals and particularly preferred nanoparticles are gold nanoparticles.

When the surface is a nanoparticle, in some embodiments of the invention, the nucleophile encapsulates the nanoparticle during treatment of the nanoparticle with the mixture comprising the sulfur-containing compound and the nucleophile.

For example, aqueous suspensions of approximately 40 nm gold nanoparticles treated with CS₂ and the nucleophile tetra (N-methyl)aminomethyl resorcinarene at millimolar concentrations were encapsulated by the resulting carbodithioate ligand. This encapsulation enabled the nanoparticles to be extracted from an aqueous phase into dichloromethane. The ability to be extracted out of the aqueous phase was only seen with encapsulated nanoparticles. No extractions occurred in controls made without CS₂ or without tetra (N-methyl)aminomethyl resorcinarene. Examples of nanoparticles capable of being encapsulated include metals, thiophilic semiconductors, and gold.

Nucleophiles of the invention include nitrogen-bearing compounds and sulfur-containing compounds.

Nitrogen-containing nucleophiles of the invention include amines or amides wherein the amines are selected from primary amines, secondary amines, heterocyclic amines, silyl amines or metal salts thereof and the amides are selected from primary amides, secondary amides and metal salt derivatives thereof.

Examples of heterocylic amines include pyrroles, diazoles, triazoles, tetrazoles, pyridones, benzoannulated derivatives, pyrazoles, imidazoles, indoles, benzimidazoles, purines, and metal salt derivatives thereof.

Nitrogen-containing nucleophiles of the invention further include dimethylamine, diethylamine, diisopropylamine, dibutylamine, didecylamine, dipicolylamine, diethanolamine, di(hexaethyleneglycol)amine, morpholine, pyridine, proline and oligopeptides bearing N-terminal prolines, piperazinyl terpyridine, nortriptylene, methamphetamine, reductive amination products of oligosaccharides with primary amines, biotin hydrazide, hexamethyldisilazane, and oligo(ethyleneglycol)diamines conjugated to molecular recognition elements such as folic acid or pteroic acid.

Sulfur-containing compounds which are nucleophiles of the invention include inorganic sulfide, thiols, thioacids, or sulfide-treated surfaces.

Suitable solvents of the invention include aqueous and organic solvents. An example of a preferred suitable organic solvent is an alcohol such as methanol, ethanol, n-propyl alcohol, and isopropyl alcohol.

Carbodithioate ligands of the invention include

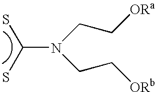

wherein R^{a} and R^{b} are independently selected from —H, alkyl, acyl, aryl or heteroaryl. The invention further includes such carbodithioate ligands bonded to surfaces of the invention.

Carbodithioate ligands of the invention further include the structure above with R^{a} and R^{b} independently selected from one of the following:

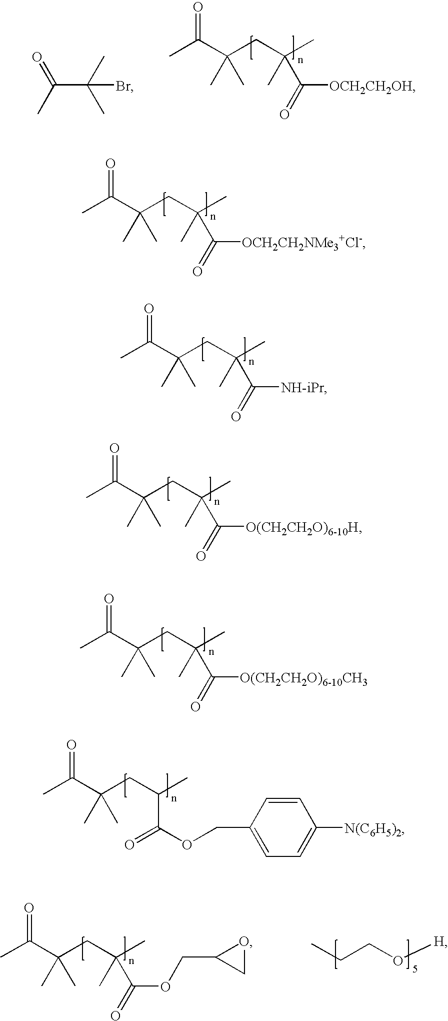

where is an integer from 1 to 99. The invention also includes such carbodithioate ligands bonded to surfaces of the invention.

Carbodithioate ligands of the invention further include the following:

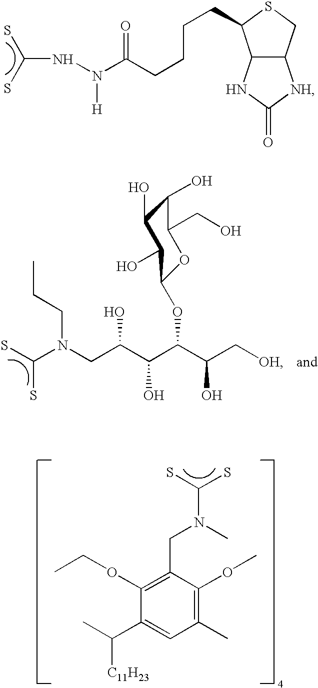

Additional carbodithioate ligands of the invention are ligands having the chemical structure

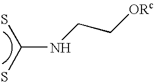

wherein R^{c} is selected from

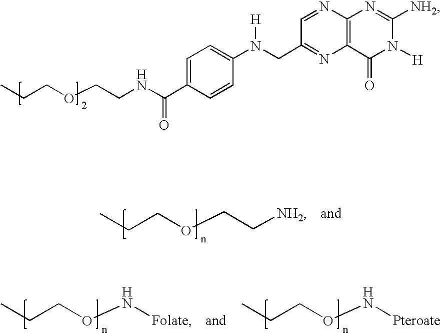

where pteroate is 2-[4-[(2-amino-4-oxo-1H-pteridin-6-yl)methylamino]benzoate], folate is the γ-glutamyl derivative of pteroate, and n=1-99.

Carbodithioate of the invention include ligands having the chemical structure:

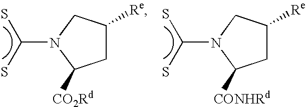

where R^{d} is selected from CH₃, an amino acid, or an oligopeptide, and R^{e} is selected from hydrogen, OH, alkyl groups, and alkoxyl groups, and the invention also includes surfaces bonded to such ligands.

The ligands of the invention are capable of encapsulating surfaces of the invention in many embodiments. For example, the ligand

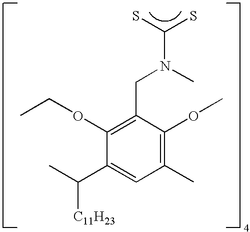

may be used to encapsulate surfaces that are nanoparticles.

The invention includes embodiments directed to methods for preparing ligands capable of molecular recognition. The surface-bound carbodithioate made according to methods of the invention may interact with the target molecular or biomolecular analyte to produce a response that can be sensed analytically. Analytical techniques used to sense the analyte may be optical or mass-based sensing. Examples of optical analytical techniques include surface plasmon resonance and surface enhanced raman scattering (SERS). Quartz crystal microbalance is a mass-based sensing technique. Thus, the surface-bound carbodithioate ligand acts as a recognition element. Such ligands may be stable under biologically relevant conditions.

In one embodiment of the invention, metal nanoparticles such as gold nanoparticles with specific plasmon resonances may be functionalized with carbodithioate-appended ligands according to the invention which, in turn, may have an affinity for biomolecular species such as peptides, small proteins, DNA, carbohydrates, oligonucleotides, and bioactive natural products. This functionalization may occur by passivating the nanoparticle with a mixture of a carbodithioate ligand not used for molecular recognition and a carbodithioate ligand capable of molecular recognition. Passivation can be extended with carbodithioates intended to block nonspecific protein adsorption, such as with di(hexaethyleneglycol) dithiocarbamate.

Examples of carbodithioates not used for molecular recognition include dimethyl dithiocarbamate, di(2-hydroxyethyl)-dithiocarbamate, and di(hexaethyleneglycol)-dithiocarbamate. Examples of carbodithioate ligands capable of molecular recognition include biotin, carbohydrates, oligopeptides, vitamin-derived moieties such as pteroate and folate, and synthetic metal ion receptors such as crown ethers and terpyridines. A particular example is N-(4-aminoterpyridinyl)-piperazinyl dithiocarbamate.

Carbodithioate ligands of the invention may be used to coat, for example, gold-coated substrates, such as those used in surface plasmon resonance (SPR) and quartz crystal microbalance (QCM) detectors, for high-throughput bioassays or pathogen detection.

The ligands used for biosensing applications of the instant invention are more stable than corresponding ligands made by prior art thiols due to the greater stability of the carbodithioate ligand-bonded surfaces of the invention. For example, under aqueous pH ranging from 1 to 12, a carbodithioate ligand of the invention, for example, reveals minimal changes to its spectral profile even after one week of immersion at ambient temperatures (see example 4). Other stresses have similarly shown the carbodithioate ligands of the invention to be more stable than thiols bonded to surfaces.

“Functionalization” of a surface refers to the act of attaching carbodithioate ligands onto a substrate for the purpose of providing one or more specific functions, such as recognition by a complementary protein or cell-surface receptor, resistance against nonspecific protein adsorption, or generation or amplification of an optical signal or signature. Preferred surfaces of this embodiment include metal nanoparticles. A particularly preferred surface is a gold nanoparticle. Functionalization can be achieved by attaching a carbodithioate ligand which can support one or more of the functions above, and/or passivating the surface with a chemically or biologically inert ligand.

An inert ligand is one in which no appreciable chemical or biomolecular adsorption or reaction takes place as determined by a suitable analytical technique. Examples of carbodithioates which are likely to be biologically inert include di(2-hydroxyethyl)-dithiocarbamate and di(hexaethyleneglycol)-dithiocarbamate.

A functional ligand is one which is capable of specific activity and/or molecular recognition. Examples of ligands which have been attached to surfaces as carbodithioates and are capable of biological function or recognition include folate and pteroate derivatives, biotin hydrazide, carbohydrates and oligosaccharides, and cell-penetrating oligopeptides.

Molecular recognition events may be determined with a suitable analytical technique such as SERS or SPR. Upon a binding event, SERS spectra will show a characteristic peak or a change in peak frequencies based on the ligand, surface, and molecular analyte used. Alternatively, one may use SERS difference spectroscopy or a combination of SERS, SERS difference spectroscopy or a combination thereof.

Because of the strong affinity carbodithioate ligands have for surfaces of the invention, such as gold nanoparticles, such ligands are particularly suited for functionalizing SERS-active sites.

In another embodiment of the invention, methods for preparing core-shell materials comprising encapsulating a nanomaterial with a carbodithioate ligand are provided. In this embodiment, the encapsulated nanomaterial may be prepared according to the invention. It is extracted into an organic solvent such as a nonpolar solvent. Examples of non-polar solvents include toluene, dichloromethane, and dichlorobenzene.

The extracted and encapsulated nanomaterial is treated with an organometallic compound to form a core-shell nanomaterial. Examples of organometallic compounds include Fe(CO)₅ and Fe(acetylacetonate)₃. The nanomaterial may alternatively be heated to temperatures over 200° C.

The resulting core-shell material contains a metal or metal oxide shell, which derives from the organometallic compound, around the nanomaterial which may be a nanoparticle or a nanorod. Preferred nanoparticles and nanorods are gold. If iron is used as the metal in the organometallic compound, the nanorod will be magnetic and will produce a strong absorption in the NIR region. Such core-shell nanomaterials could be used as contrast agents for biomedical imaging applications.

In a yet another embodiment of the invention, methods for preparing imaging agents are provided wherein the surface of a nanoparticle is passivated and functionalized. In this embodiment, the nanoparticle surface is functionalized with a combination of biologically active nucleophiles and biologically inactive nucleophiles. Preferred nanoparticles of this embodiment are gold nanoparticles including gold nanorods. Optionally, the gold nanorod may be coated with a cationic surfactant such as cetyltrimethylammonium bromide. Other nanoparticles include CdSe and iron oxide.

Surface passivation may occur by exchanging the surfactant molecules using standard chemical techniques with carbodithioate ligands. This may be achieved, for example, by condensing a sulfur-containing compound and a nucleophile in a suitable solvent onto the nanoparticle thereby forming a carbodithioate ligand. A preferred sulfur-containing compound is CS₂, a preferred nucleophile is oligo(ethyleneglycol)amine, and water is a preferred solvent.

Biologically inert ligands are those that do not interact appreciably with biomolecular species whereas biologically active ligands provide the basis for a positive detection of biomolecular species.

Examples of biologically inert ligands include oligo(ethyleneglycol)amines whereas biologically active ligands would be formed from amines conjugated to, for example, pteroate or folate ligands. For example, the folate receptor is known to be over expressed in many tumor cells. Thus, such biologically active ligands may be used to label tumor cells.

Alternatively, such biologically active ligands may be used for treatment of diseased cells like, for example, cancer cells by hyperthermia. Hyperthermia is a noninvasive technique in which biological tissues are exposed to higher than normal temperatures to promote the selective destruction of abnormal cells. Moderate increases in temperature may sensitize cancer cells to cytotoxic agents by increasing membrane permeability and lowering hydrostatic pressure. In addition, mild hyperthermia can induce the production of heat shock proteins and other immunostimulants, trigger dysfunctional cellular metabolism, and promote the onset of acidosis or apoptosis. Moreover, tumor cells are considered to be more susceptible to hyperthermic effects than healthy cells because of their higher metabolic rates.

In one embodiment methods of promoting photoinduced cell injury or death using the surface-bound carbodithioate ligands of the present invention are provided. In one method, a cell is contacted with a surface-bound carbodithioate ligand, where the carbodithioate-ligand is capable of molecular recognition. Non-limiting examples of ligands capable of molecular recognition may be folates, pteroates, antibodies (including polyclonal and monoclonal antibodies), peptides and oligopeptides, growth factors, steroids or hormones. These functional ligands may recognize proteins and/or receptors on the external cell surface and upon contact with the cell, may adsorb, bind or associate with the cell. The surface-bound carbodithioate ligand may or may not be internalized into the cells. It may actually be advantageous for the surface-bound carbodithioate ligand to be bound to the external surface of the cell as hyperthermia may occur at lower irradiation power and shorter exposure times.

In an illustrative embodiment, the cell comprises folate receptors and the carbodithioate ligand comprises folate or pteroate. In an alternate illustrative embodiment, the cell comprises HER2/NEU receptors and the carbodithioate ligand comprises a monoclonal antibody such as trastuzumab, commonly known as the anticancer drug Herceptin®. Other examples of external cell surface receptors that may be targeted may be, but not limited to, EGF receptors, PDGF receptors, IGF receptors, TGF-α receptors, ErbB receptors or peptide receptors. Alternatively, antibodies to cell surface proteins may also be used.

After contacting the cell with the surface-bound carbodithioate ligand, the cell is exposed to irradiation. When the surface is nanoparticle, irradiation of the surface-bound carbodithioate increases the temperature of the nanoparticle and thus increasing the temperature at the cell. Photoinduced cell injury or death results from the irradiation of the cell in the presence of the carbodithioate ligand. The conditions for irradiation will vary based on the source of the irradiation, the location of the ligand, the time of exposure and the power. Most commonly, the irradiation source may be a focused continuous wave laser beam or a femtosecond-pulsed laser beam. The laser beam may be tuned to the nanorod plasmon resonance peak. For example, the nanorod plasmon resonance peak may be from 750 nm to about 850 nm. For gold nanoparticles, the resonance peak is about 765 nm. The average power of the irradiation may be from about 0.6 mW to about 60 mW. When the ligands are fully internalized greater power levels may be required to induce cell injury or death in contrast to when the ligands are on the external cell surface. The time frame for the irradiation may be from about 1 second to about 90 seconds. Irradiance exposure may be a single scan or it may be multiple scans. It will be appreciated that the irradiance needed for cell damage may depend on net exposure times and can be further reduced for longer irradiation intervals.

The term “alkyl” is intended to include linear, branched, or cyclic hydrocarbon structures and combinations thereof. Examples of alkyl groups include methyl, ethyl, propyl, isopropyl, butyl, s- and t-butyl and the like. Cycloalkyl is a subset of alkyl and includes cyclic hydrocarbon groups of from 3 to 13 carbon atoms. Examples of cycloalkyl groups include cyclopropyl, cyclobutyl, cyclopentyl, norbornyl, adamantyl, menthyl, and the like. As used herein, alkyl refers to alkanyl, alkenyl and alkynyl residues; it is intended to include cyclohexylmethyl, vinyl, allyl, isoprenyl and the like.

The surface-bound carbodithioate ligands may be used to treat patients with a tumor in order to decrease the size or eliminate the tumor altogether. It is known in the art that cancer cells overexpress a number of different external cell surface receptors. Different receptors are overexpressed for different cancers. For certain types of breast cancers, the HER2/NEU receptor is overexpressed. In other forms of cancer, folate receptors are overexpressed. If the patient has a form of cancer that overexpresses a specific receptor, the patient may be administered a therapeutically effective amount of a metal nanoparticle-bound carbodithioate ligand where the carbodithioate ligand is capable of molecular recognition of the overexpressed receptor. For example, if the cancer and/or tumor overexpresses a folate receptor, the ligand may comprise folate or pteroate. Alternatively, if the patient has a breast cancer that overexpresses the HER2/NEU receptor, then the ligand may be trastuzumab. A therapeutically effective amount will vary with the type of cancer and the size of the tumor. It is well within the ability of the skilled clinician to determine the amount of ligand required. The ligand may be administered systemically or it may be administered directly to the site of the tumor.

After administering the metal nanoparticle-bound carbodithioate ligand, the tumor is irradiated as described above. Several courses of treatment may be required. Alternatively, the methods of the present invention may be used in a combination therapy where, after irradiation, a known cytotoxic drug is administered to the patient to aid in eliminating the tumor.

“Acyl” refers to a straight, branched or cyclic configuration of carbon atoms, typically from 1 to 20, or a combination of any such configurations, attached to the parent structure through a carbonyl functionality. Such acyl groups can be saturated or unsaturated, and aromatic or non-aromatic. One or more carbons in the acyl group may be substituted with nitrogen, oxygen or sulfur as long as the point of attachment to the parent remains at the carbonyl. Examples include acetyl, benzoyl, propionyl, isobutyryl, t-butoxycarbonyl, benzyloxycarbonyl and the like.

“Aryl” and “heteroaryl” mean a 5- or 6-membered aromatic or heteroaromatic ring containing 0-3 heteroatoms selected from O, N, or S; a bicyclic 9- or 10-membered aromatic or heteroaromatic ring system containing 0-3 heteroatoms selected from O, N, or S; or a tricyclic 13- or 14-membered aromatic or heteroaromatic ring system containing 0-3 heteroatoms selected from O, N, or S. Examples of aromatic compounds which may be used to form aryl or heteroaryl groups include benzene, naphthalene, indane, tetralin, fluorene, imidazole, pyridine, indole, thiophene, benzopyranone, thiazole, furan, benzimidazole, quinoline, isoquinoline, quinoxaline, pyrimidine, pyrazine, tetrazole and pyrazole.

“Biologically relevant conditions” means those conditions, such as temperature and pH, that would be found in biological fluids relevant for medical treatment or diagnostics. Such fluids include blood and gastrointestinal fluid.

“Molecular recognition” refers to the capacity of ligands (i.e., recognition elements) to bind analytes or other molecules including biomolecules such as proteins with high levels of specificity and/or avidity.

“Passivation” refers to the lowering of a surface's chemical reactivity toward nonspecific adsorption. This includes the adsorption of carbodithioate ligands which provide resistance against nonspecific protein adsorption.

“Stable” means that a carbodithioate ligand on a surface does not appreciably dissociate under biological conditions during the timespan of an analytical test design for sensing applications.

The term “xanthates” means salts of xanthic acid of the general formula ROC(S)SH where R is alkyl. One typical xanthic acid is where R is ethyl.

The following examples illustrate several embodiments of the invention and in no way meant to be limiting.

### Preparation of Chemical Reagents:

Spectrophotometric grade CS₂ (Sigma-Aldrich) was freshly distilled from CaH₂ just prior to use. Dimethylamine, diethylamine, dibutylamine, didecylamine, diisopropylamine, piperidine, and morpholine (Aldrich) were used without further purification. In the case of dimethylamine, neat samples could be obtained by condensation at −78° C.

### Roughened Au Substrates:

Au foil (0.1 mm, Alfa Aesar) was cut into 6×6 mm squares and annealed by a propane torch for 2-3 min, sonicated for 10 min in deionized water, then roughened using a potentiostat (Princeton Applied Research 273A) in 0.1 M KCl. The parameters for electrochemical roughening were as follows: (i) initial potential (E1) of −0.3 V, (ii) a delay time (D1) of 30 sec; (iii) ramping the potential at a rate of 500 mV/sec to an upper limit (E2) of 1.2 V; (iv) a delay time (D2) of 1.3 sec, (v) decreasing the potential at a rate of 500 mV/sec to E1. The oxidation-reduction process was repeated for 25 cycles, in accord with the protocol of Weaver and coworkers (J. Electroanal. Chem. 1987, 233, 211). All voltages were referenced against a saturated calomel electrode (SCE).

### Smooth Au Substrates:

Thin glass cover slips (Corning, 18′18 mm²) were coated with a 10-nm Cr adhesive layer and 50-nm Au film by thermal evaporation, and used immediately after preparation.

### Double Layered Nanoporous Gold Leaf:

Gold leaf was treated in HNO₃ (70%) 30 min, then transferred to mercapto-modified glass slides. Samples were then heated at 110° C. for 30 min. Samples were treated in 10 mM NaI/I₂ for 20 min. Then transferred second layer of nanoporouse gold leaf was heated at 110° C. for 10 min and treated in 10 mM NaI/I₂ for 20 min. Colloidal Au nanoparticles in aqueous suspension (British Biocell International, EM.GC40, ˜1011 particles/mL) were treated with a mixed-bed ion-exchange resin (Amberlite MB-3, Mallinckrodt) for 30 minutes to minimize the presence of electrolyte in solution.

### Cell Culture.

KB cells and NIH/3T3 cells were kindly provided by Dr. Philip Low and Dr. Stephen Konieczny (Purdue University). All cells were cultured at 37° C. in a humidified atmosphere containing 5% CO₂ and grown continuously in folate-deficient RPMI 1640 medium (Invitrogen) containing 10% fetal bovine serum (FBS, Sigma) and 1% penicillin-streptomycin (Invitrogen). In a typical experiment, a 1-mL suspension of KB cells (10⁵ cells/mL) was plated onto a coverslip-bottomed Petri dish (MatTek), grown for 2-3 days, then treated with an aliquot of F—NRs (100 μL) and maintained at 37° C. with periodic monitoring.

### Two-Photon Luminescence Imaging (TPL).

A femtosecond (fs) Ti:Sapphire laser (Mira 900, Coherent, Santa Clara, Calif.) was used to generate laser pulses with a duration of 200 fs at a repetition rate of 77 MHz. The TPL excitation power was 0.75 mW at the sample. The laser beam was directed into a scanning confocal microscope (FV300/IX70, Olympus, Melville, N.Y.) equipped with a 60× water-immersion objective (NA 1.2). Consecutive real-time images (movies) were recorded at a speed of 0.6 frames per second over a period of 59.8 s, using the transmission signal to visualize the KB cells.

### EXAMPLE 1

### Carbodithioate Ligands Formed on Au Surfaces

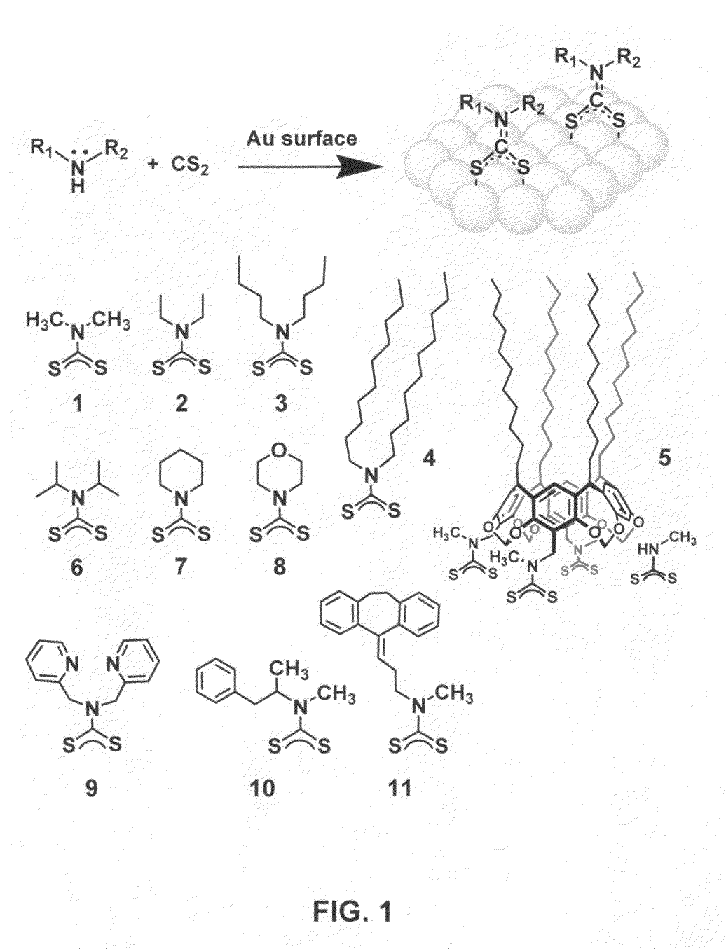

The ligands of FIG. 1 were readily formed by immersing gold substrates in solutions containing an equimolar ratio of CS₂ and the corresponding amine under slightly basic conditions, as well as by using a 2:1 ratio of CS₂ to amine. For example, a 10% solution of CS₂ in methanol (1 mL) was treated dropwise with one molar equivalent of secondary amine dissolved in the same solvent (1 mL), followed by vortex mixing for 30 sec. The final concentration of dithiocarbamate was 0.42 M. Au substrates were introduced and soaked for variable periods, then rinsed twice in pure methanol and dried in air. The structure of the corresponding amine can be derived by substituting a hydrogen from the —CS₂ moieties for each figure. Ligand 5, the tetradithiocarbamoyl derivative of tetra(N-methyl)aminomethyl resorcinarene, contains four —CS₂ units and the corresponding amine had four hydrogens in place of those units.

The reactions to form the ligands in FIG. 1 were performed separately in one-pot reactions where CS₂ and the amine corresponding to each structure were combined in water, methanol, ethanol, or mixtures thereof.

Contact angle measurements of carbodithioate-functionalized gold surfaces revealed marked changes in their wetting properties: smooth gold substrates coated with dimethyl dithiocarbamate (ligand 1) (θₐᵥ=60°) are more hydrophilic than bare gold (θₐᵥ=80°), whereas those coated with dibutyl and didecyl dithiocarbamates (see ligands 3 and 4) are more hydrophobic (θₐᵥ=107° and 108°, respectively). By comparison, substrates treated with dialkylamines in the absence of CS₂ did not exhibit significant changes in wetting behavior after rinsing.

### EXAMPLE 2

### Gold Nanoparticles

Carbodithioates were assembled on colloidal gold nanoparticles by suspending aqueous suspensions of 40-nm Au particles treated with CS₂ and tetra(N-methyl)aminomethyl resorcinarene (TMAR) at millimolar concentrations. The aqueous suspension of Au colloid (1 mL) was treated with a mixed-bed ion-exchange resin (MB-3), then decanted and mixed vigorously with a 1 mM solution of CS₂ in THF (1 mL). A 1 mM solution of TMAR in THF (1 mL) was added, and the solution was agitated vigorously by vortex mixing for another 5 minutes. Addition of CH₂Cl₂ (1 mL) resulted in phase separation, with extraction of the nanoparticles to the organic phase. No extractions occurred in the absence of CS₂ or TMAR.

### EXAMPLE 3

### Characterization Studies

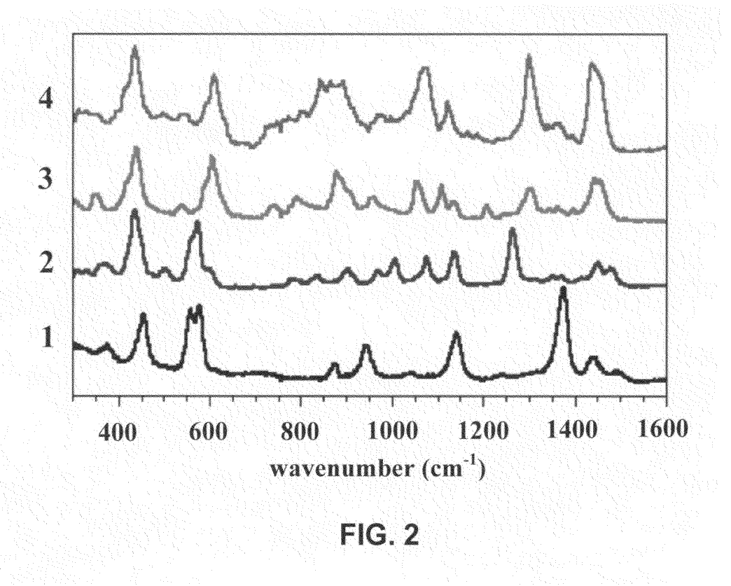

A carbodithioate assembly on a roughened Au substrate was further characterized using surface-enhanced Raman spectroscopy (SERS; see FIG. 2). The SERS spectra of ligands 1-4 of example 1, formed by in situ condensation of amines with CS₂, were found to be nearly identical with those generated from preformed dithiocarbamate sodium salts, thus providing confirmation of structure.

Vibrational modes were assigned according to density functional theory (DFT) calculations: Raman frequencies were calculated for ligands 1 and 2 bonded to a cluster of gold atoms (1-3 atoms) using the B3LYP method and LANL2DZ basis set. The calculated values correlated well with the experimental SERS data, most notably for peak frequencies at 430-450 cm⁻¹ (I) and 540-600 cm⁻¹ (II). These vibrational bands correspond with symmetrically coupled C—S stretching and N-alkyl bending (scissoring) modes of the carbodithioate moiety anchored to the metal surface (see FIG. 4). Moreover, the SERS bands at 1450-1475 cm⁻¹ associated with C—H bending modes increased in prominence with hydrocarbon chain length.

### EXAMPLE 4

### Robustness Studies

Roughened Au substrates functionalized with ligands 1 and others functionalized with ligand 2 were exposed to aqueous solutions ranging from pH 1 to pH 12 and monitored by SERS. The data revealed minimal changes in spectral profile after one week of immersion at ambient temperature. In a second instance, ligand-coated substrates were immersed for one week in ethanolic solutions of dodecanethiol, again with minimal perturbations to their SERS spectra.

A smooth Au surface coated with dibutyl dithiocarbamate (ligand 3) was exposed to a millimolar solution of 2-mercaptoethanol for 24 hours, a condition known to completely displace alkanethiol monolayers. Minimal change in contact angle was observed (Δθₐᵥ<3°), and analysis by x-ray photoelectron spectroscopy (XPS) showed that the S:N mole ratio remained unchanged at 2.14 to 1. The limits of thermal stability under aqueous conditions were also examined, with a decrease in contact angle finally observed after 12 hours at 85° C.

### EXAMPLE 5

### Functionalization of SERS-Active Nanoporous Gold

A nanoporous gold substrate was prepared by etching white gold leaf in concentrated nitric acid, similar to the process described by Erlebacher and coworkers (Adv. Mater. 16, 1897-1900 (2004)). The nanoporous gold leaf was rinsed with deionized water, bonded onto a glass slide modified with mercaptopropyltrimethoxysilane, then further etched with iodine and potassium iodide to yield a substrate that was highly SERS-active at an excitation wavelength of 785 nm. Depositing a second layer of nanoporous gold leaf further increased the activity and reproducibility of the SERS signal intensities.

The substrate was then functionalized with carbodithioate ligands using a two-step process, on the premise that the SERS-active sites are recessed within the nanoporous substrate and are only functionalized at a late stage of the coating process. The first step involved passivating the substrate with simple carbodithioate ligands with no recognition capabilities. Carbodithioates used were dimethyl-dithiocarbamate, di(2-hydroxyethyl)-dithiocarbamate, and di(hexaethyleneglycol)-dithiocarbamate.

The ligand concentration and soaking period was optimized for maximum surface coverage without producing any vibrational signals related to the dithiocarbamate ligands themselves. The second step involved adding the dithiocarbamate ligand N-(4-aminoterpyridinyl)-piperazinyl dithiocarbamate, which can serve as a synthetic receptor for Zn ions. A higher ligand concentration and a longer soak time was used to drive the ligands into the SERS-active sites. This two-step process ensures that the molecular analyte recognition should only occur at SERS-active sites. The molecular recognition event was detected by observation of a characteristic Raman signal, by SERS difference spectroscopy, or a combination thereof.

### EXAMPLE 6

### Prophetic Example of Synthesis of Core-Shell Nano Materials

Calixarene-based multivalent dithiocarbamates may be used as surfactants, specifically when formed by the in situ condensation of CS₂ and Tetra(N-methyl)aminomethyl resorcinarene (TMAR) and can further be used to extract colloidal Au nanoparticles (40 nm) and Au nanorods (15×50 nm) into nonpolar organic solvents such as toluene and dichloromethane, which can then be transferred to other solvents such as dichlorobenzene. The superior surfactant properties of calixarenes and particularly resorcinarenes have been previously described for dispersing nanoparticles (Wei, A., ChemComm, 1581-1591 (2006); Wei, A.; Kim, B. “Nanoparticle Arrays and Sensors Using Same.” U.S. Pat. No. 6,899,947, issued May 31, 2005) which are incorporated herein by reference.

Nanorods encapsulated in TMAR-based DTC surfactants can be heated to over 200 degrees Celsius for short periods of time without degradation or precipitation. This dispersion control enables the synthesis of a core-shell nanomaterial. For example, an organometallic precursor (e.g., Fe(CO)₅ or Fe(acetylacetonate)₃) can be injected into the hot nanorod suspension at high temperatures to form an iron or iron-oxide shell around the nanorod core, producing a magnetically active nanorod with strong absorption in the NIR region.

### EXAMPLE 7

### Forming Functionalized Nanoparticles for Use as Biological Imaging Agents

Gold nanorods coated with cetyltrimethylammonium bromide (CTAB, a cationic surfactant) can be passivated using simple surfactant exchange with DTC ligands, formed by in situ condensation of oligo(ethyleneglycol)amines with CS₂ in aqueous solutions. Surfaces comprised of such molecules are expected to resist nonspecific protein adsorption. The passivated nanorods can be purified by dialysis using a semi-permeable membrane to remove excess surfactant. These nanorods are anticipated to be biologically inert and have a long circulation half-life, whereas CTAB-coated nanorods would be expected to be rapidly internalized via a nonspecific cell uptake mechanism. Iron oxide and CdSe nanoparticles are also amenable to functionalization with hydrophilic DTC ligands, and can form stable suspensions in aqueous solutions.

Nanoparticles can be functionalized with biologically active ligands by sequential conjugation reactions. In one instance, a 1-mL suspension of gold nanorods (ca. 10⁹ particles) was treated with 8.8 mg of oligo(ethyleneglycol)diamine (n˜18, ca. 28 μmol) and 100 μL of a saturated CS₂ solution (ca. 2.8 μmol) to produce carbodithioate ligand at an assumed final concentration of 2.8 mM. The mixture was stirred overnight, and the excess ligand was separated from the functionalized nanorods by dialysis using a cellulose membrane with MWCO of 6000-8000. The amine-coated nanorods were treated with 50 μL of a folate-NHS solution (10 mM in DMSO) with overnight stirring. The resulting folate-conjugated nanorods were separated from the excess folate by membrane dialysis.

### EXAMPLE 8

### Prophetic Example on Using Functionalized Ligands

Nanoparticles can be functionalized with biologically active ligands at controlled densities using a binary surfactant system. In one instance, oligo(ethyleneglycol)amines conjugated with pteroate or folate ligands can be combined with inert oligo(ethyleneglycol)amines in the presence of CS₂, then introduced to nanoparticles with the expectation that their surfaces will be completely passivated by carbodithioate units, with a statistical ratio of biologically active and inert ligands. This stoichiometric control is useful for tuning the binding avidity of nanoparticles; for example, the surface density of folate ligands can be optimized for labeling tumor cells which over express the folate receptor, while minimizing adventitious binding to healthy cells which display a normal level of this surface protein.

### EXAMPLE 9

### Carbodithioate Formation by CS₂ and Biotin Hydrazide on Gold Surface

5 μL CS₂ was dissolved in 0.5 mL DMSO. 5.4 mg biotin hydrozide was dissolved in 0.5 mL DMSO, and added to CS₂ solution dropwise under stirring. Au substrates were introduced and soaked for 10 minutes, then rinsed twice in pure methanol and dried in air.

### EXAMPLE 10

### Preparation and Characterization of Folate-Conjugated Gold Nanorods (F—NRs)

Gold nanorods were prepared using seeded growth conditions in the presence of cetyltrimethyl-ammonium bromide (CTAB) and silver nitrate (Sau, T. K. & Murphy, C. J. (2004) Langmuir 20, 6414-20), then treated with sodium sulfide 30 min after injection of the seed solution to arrest further growth and changes in their optical resonances. The nanorods were centrifuged and redispersed in deionized water two times (24,000×g, 5 min per cycle) to remove most of the CTAB and residual metal sulfide, then diluted to an optical density (O.D.) of 1.0-1.2. Particle size analysis by transmission electron microscopy indicated a mean length and aspect ratio of 46.5 nm and 3.7, respectively.

Amine-terminated oligoethyleneglycol chains were tethered onto nanorods by in situ dithiocarbamate formation as described herein. An aqueous suspension of CTAB-coated nanorods (3 mL, O.D. 1.0) was treated with a mixed-bed ion-exchange resin (Amberlite MB-3, Sigma) for several hours at room temperature to remove unassociated surfactant and other ions, then decanted and treated while stirring with a 10-mM solution of O,O′-bis(2-aminoethyl)octadecaethylene glycol (Fluka) adjusted to pH 9.5 (1 mL), followed by a saturated solution (28 mM) of freshly distilled CS₂ (0.1 mL) for in situ DTC formation. The mixture was stirred for 12 hours and then subjected to membrane dialysis for 2 hours (MWCO 6000-8000) to produce stable dispersions of amino-OEG-coated nanorods. Exhaustive dialysis is necessary for the complete removal of CTAB in order to minimize the nonspecific cell uptake of nanorods. The amine-coated nanorods were then treated with a 10-μM DMSO solution of N-hydroxysuccinimidyl folate (folate-NHS) (0.2 mL) prepared according to literature procedure (Lee, R. J. & Low, P. S. (1994) J. Biol. Chem. 269, 3198-204), followed by additional dialysis to yield a stable dispersion of F—NRs with a final absorption maximum at 765 nm and optical density close to 1. TPL correlation spectroscopy was used to measure the concentration and mean hydrodynamic diameter of the F—NRs, which were determined to be 7.5 nM and 81.5 nm, respectively. It should be mentioned that the absorption spectra of the nanorods prepared were not significantly affected by surface functionalization, although the longitudinal plasmon resonance is often sensitive to changes in the surface dielectric.

It is essential to maintain nanorod dispersion stability during DTC formation and surface functionalization. Exposing the nanorods to ion-exchange resin for more than a few hours causes precipitation of the nanorods. Introducing small quantities (˜10 ppm) of an anionic polyelectrolyte such as polystyrenesulfonate can be used to coat the nanorods in order to maintain a negative surface charge density during CTAB exchange and depletion. Ion-exchange resins can also alter the pH of the nanorod dispersion solution, which has a strong effect on the rate of DTC formation. If the pH of the nanorod dispersion is too low, an alternate procedure which ensures the formation of OEG-DTC ligands is to mix the OEG-diamine and CS2 as a concentrated (˜10 mM) solution at pH>12 for 2 hours, followed by addition to nanorod dispersions until a final pH of about 8 to about 9 is achieved.

### EXAMPLE 11

### Laser Scanning and Fluorescence Imaging of F—NR Mediated Photothermal Damage

KB cells incubated with F—NRs were rinsed with fresh RPMI 1640 medium prior to scanning with the Ti:sapphire laser, which could be readily switched between fs-pulsed and cw mode. Cells in a 39.3×39.3 μm² area were scanned continuously for 81.4 s (49 scans with 1.66 s per scan). Cells were irradiated at 765 nm using either mode under constant average power, ranging from 0.75 to 60 mW at the sample. The mean power density was calculated by dividing the average laser power with the scanning area. The focal spot area was calculated as πd²/4, where d=0.61λ/NA is the full width at half maximum of the beam waist. Each scan was digitized into 512×512 pixels (pixel area=77×77 nm²; exposure time=6.3 μs per pixel per scan). The exposure time for a nanorod in a single scan was approximated as (focal spot area/pixel area)×6.3 μs=0.126 ms; the total exposure time was calculated as 0.126 ms×49 scans=6.174 ms.

Cell death was determined after treatment with 2 μL of 0.05% ethidium bromide, a nuclear staining dye used to test membrane integrity (Wyllie, A. H. et al. (1980) Int. Rev. Cytol. 68, 251-305). Cell viability was determined by treatment with calcein AM (final concentration 2.5 μM), a dye that labels live cells (Pitsillides, C. M. et al. (2003) Biophys. J. 84, 4023-4032; Loo, C. et al. (2005) Nano Lett. 5, 709-711). In some cases, the plasma membrane of KB cells was stained by treatment with folate-Bodipy (final concentration 100 nM), prepared according to literature procedures (Sandoval, R. M. et al. (2004) Am J Physiol Cell Physiol 287, C517-C526). For monitoring the integrity of actin filaments, KB cells were transfected with plasmids encoded for β-actin conjugated to green fluorescent protein (actin-GFP) using a commercial transfection agent (FuGene 6, Roche), three days prior to photothermal treatment. For pharmacological disruption of actin filaments, KB cells were also incubated with cytochalasin D (final concentration 5 μg/mL) for 2 h prior to observation. Fluorescence was excited by a 488-nm Ar⁺ laser, with 37.5 μW at the sample.

### EXAMPLE 12

### Selective Uptake and Intracellular Trafficking of F—NRs in KB Cells

Folate-mediated targeting proved to be a useful mechanism for the selective delivery and uptake of gold nanorods. KB cells (a tumor cell line known to overexpress the high-affinity folate receptor, FR) were treated with a suspension of F—NRs and observed by TPL microscopy to be densely coated after 6 h incubation. F—NRs bound to the surface of the KB cell membrane could be dislodged by washing the cells with a pH 3.3 buffer. F—NRs were also applied to cultured NIH-3T3 cells having low FR expression with little binding observed after 6 h, confirming the receptor-targeted nature of nanorod adsorption. The receptor-bound F—NRs were very slowly internalized, but observed to be fully translocated to the perinuclear region after many hours.

### EXAMPLE 13

### Site-Dependent Photothermal Effects

Cultured KB cells incubated with F—NRs were scanned continuously for 81.4 s by a tightly focused continuous-wave (cw) or femtosecond (fs)-pulsed laser beam tuned to the nanorod plasmon resonance peak (λₘₐₓ=765 nm), with average powers ranging from 0.75 to 60 mW at the sample. KB cells with either membrane-bound F—NRs or internalized F—NRs experienced extensive blebbing along the cell periphery after cw laser irradiation, and their nuclei were heavily stained by ethidium bromide (EB) indicating loss of membrane integrity. The TPL signals were also greatly diminished after cw irradiation, signifying that most of the nanorods had melted and were no longer resonant at NIR frequencies. To verify that blebbing was due to morphological changes in the membrane, a generally accepted sign of cell death, KB cells with internalized F—NRs were treated with Bodipy-conjugated folate 30 min prior to cw irradiation. The boundary of the resulting blebs was clearly fluorescent, confirming photoinduced deformation of the cell membrane.

The threshold for cell damage was found to be strongly dependent on the localization of the nanorods. For fully internalized F—NRs, an irradiation power of 60 mW was needed to induce membrane blebbing during cw laser illumination, whereas a power of 6 mW was sufficient for damage in the case of membrane-bound F—NRs. While not wishing to be bound by theory, the greater sensitivity of the latter may be due to the low thermal conductivity of the medium surrounding the membrane-bound F—NRs, which helps to sustain higher temperatures with subsequently more intense hyperthermic effects. The irradiance needed for cell damage presumably depends on net exposure times and can be further reduced for longer irradiation intervals; indeed, the fluence required for photodamage was found to be remarkably low (see below).

Tumor cells with membrane-bound F—NRs also exhibited membrane blebbing and strong EB staining in response to fs-pulsed laser irradiation, but at a reduced average power of 0.75 mW, which corresponds to a pulse energy of 9.7 pJ and a mean power density of 48.6 W/cm². The brightness of the TPL signals remained constant under fs-pulsed irradiation, indicating that the rate of heat dissipation was sufficient to prevent the nanorods from melting. In comparison, KB cells with internalized F—NRs remained viable after fs-pulsed irradiation at a higher power of 4.5 mW, as indicated by strong intracellular calcein fluorescence. This site-dependent cell kill is consistent with the observations above using cw irradiation. Finally, cells devoid of nanorods were unaffected by either cw or fs-pulsed irradiation, and cells with internalized nanorods did not exhibit signs of cytotoxicity after 24 h incubation. These controls confirm that F—NR mediated thermolysis is the primary cause of photoinduced cell death.

The susceptibility of KB cells to laser-induced damage increased dramatically upon labeling with F—NRs, particularly when localized on the cell surface. In order to determine the fluences used in F—NR mediated cell damage, the laser scanning settings used in herein study were first taken into account, which limits the total exposure time of each nanorod to approximately 6.2 ms. The threshold fluences under cw irradiation at 6 mW and fs-pulsed conditions at 0.75 mW were thus 24 and 3 J/cm², respectively. These values compare favorably with a recent study on nanorod-mediated hyperthermia induced by a widefield illumination source, for which a power density of 10 W/cm² and exposure time of 4 min was reported (Huang, X. et al. (2006) J. Am. Chem. Soc. 128, 2115-20).

The notable difference in threshold fluence for photothermolysis by cw and fs-pulsed laser irradiation may be partly due to nanorod melting from the high powers used in cw mode, but other factors may also have an impact on optothermal energy conversion. The photothermal activity of the plasmon-resonant nanorods is driven by the ultrafast thermalization of conduction electrons on the femtosecond timescale, followed by electron-phonon relaxation on the picosecond timescale with subsequent thermalization of the phonon lattice. However, saturation absorption can result in transient plasmon bleaching, whose recovery rate is also on the order of picoseconds. Plasmon bleaching is not expected to be a limiting factor when using fs-pulsed excitation with nanosecond intervals for plasmon relaxation, but it may reduce the absorption efficiency of nanorods under cw irradiation with little opportunity for plasmon recovery. Another factor which may contribute toward the nanorod's photothermal efficiency under fs-pulsed excitation is its plasmon-enhanced two-photon absorption cross section, which offers a mechanism for increasing the population and energy of photoexcited electrons in the conduction band.

1. A method for producing photoinduced injury to a cell comprising the steps of:
contacting the cell with a surface-bound carbodithioate ligand, wherein the carbodithioate ligand is capable of molecular recognition; and
irradiating the cells at near-infrared frequencies to cause photoinduced injury to the cell.

2. The method of claim 1 wherein the surface is a metal nanoparticle.

3. The method of claim 1 wherein the surface is a gold nanoparticle.

4. The method of claim 1 wherein the surface bound carbodithioate ligand adsorbs to a receptor on an external surface of the cell.

5. The method of claim 1 wherein the carbodithioate ligand comprises a carbohydrate, an oligopeptide, a peptide, a pteroate, an antibody, a growth factor, a steroid or a hormone.

6. The method of claim 1 wherein the carbodithioate ligand is an oligo(ethyleneglycol) amine conjugated with a carbohydrate, an oligopeptide, a peptide, a pteroate, an antibody, a growth factor, a steroid or a hormone.

7. The method of claim 1 wherein the cells are cancer cells.

8. The method of claim 1 wherein the cells overexpress a folate receptor, a peptide receptor or a growth factor receptor.

9. The method of claim 7 wherein the growth factor receptor is a HER2/NEU receptor, an EGF receptor, a PDGF receptor, IGF receptor, a TGF-α receptor or an ErbB receptor.

10. The method of claim 1 wherein the cells are irradiated at a wavelength of from about 750 nm to about 850 nm.

11. The method of claim 1 wherein the cells are irradiated at a power of about 0.5 mW to about 60 mW.

12. The method of claim 1 wherein the cells are irradiated for from about 1 second to about 90 seconds.

13. A method for producing photoinduced cell death in a cell, wherein the cell has an overexpression of an external receptor, the method comprising the steps of:
contacting the cell with a metal nanoparticle-bound carbodithioate ligand, wherein the carbodithioate ligand is capable of molecular recognition of the receptor; and
irradiating the cell at near-infrared frequencies to cause photoinduced cell death.

14. The method of claim 12 wherein the receptor is a folate receptor or a HER2/NEU receptor.

15. The method of claim 13 wherein the carbodithioate ligand comprises folate, pteroate or trastuzumab.

16. The method of claim 12 wherein the cell is a cancer cell.

17. The method of claim 12 wherein the metal nanoparticle is gold.

18. The method of claim 12 wherein the cell is irradiated at a wavelength of from about 750 nm to about 850 nm.

19. The method of claim 12 wherein the cell is irradiated at a power of from about 0.6 mW to about 60 mW for a time period of from about 90 seconds to about 1 second.

20. A method for treating a patient with a tumor wherein the tumor comprises cells which overexpress an external receptor, said method comprising:
administering a therapeutic amount of a metal nanoparticle-bound carbodithioate ligand, wherein the carbodithioate ligand is capable of molecular recognition of the receptor; and
subjecting the patient to near infrared irradiation at the tumor.

21. The method of claim 20 wherein the metal nanoparticle is a gold particle.

22. The method of claim 20 wherein the external receptor is a folate receptor or a HER2/NEU receptor and the carbodithioate ligand comprises folate, pteroate or trastuzumab.

23. The method of claim 20 wherein the metal nanoparticle-bound carbodithioate ligand is administered directly to the tumor;

24. The method of claim 20 wherein the irradiation has a wavelength from about 750 nm to about 850 nm, a power from about 0.6 mW to about 60 mW, and is administered for a time period of from about 1 second to about 90 seconds.

25. The method of claim 20 further comprising the step of administering a cytotoxic drug to the patient after irradiation of the tumor.
